(19) United States
(12) Patent Application Publication
(10) Pub. No.: US 2025/0302547 A1
(43) Pub. Date: Oct. 2, 2025

(54) Technique For Supporting Users In An Operating Room By Triggering Feedback Regarding Medical Instruments

(71) Applicant: Stryker European Operations Limited, Carrigtwohill (IE)

(72) Inventors: Renu Khorwal, New Delhi (IN); Saurabh M. Dube, Mumbai (IN); Ravi K. Raina, Noida (IN); Inderjeet S. Bedi, Pratap Pura (IN); Oliver Weede, Freiburg (DE); José Luis Moctezuma de la Barrera, Freiburg (DE); Yannik Guth, Freiburg (DE)

(73) Assignee: Stryker European Operations Limited, Carrigtwohill (IE)

(21) Appl. No.: 19/183,374

(22) Filed: Apr. 18, 2025

Related U.S. Application Data
(63) Continuation-in-part of application No. 18/761,420, filed on Jul. 2, 2024.

(30) Foreign Application Priority Data
Jul. 4, 2023 (EP) 23183394.8

(51) Int. Cl. A61B 34/20; A61B 34/00; A61B 34/10; A61B 90/00

(52) U.S. Cl. CPC A61B 34/20; A61B 34/10; A61B 34/25; A61B 90/36; A61B 2034/107; A61B 2034/2046; A61B 2034/254; A61B 2090/364

(57) ABSTRACT

A computer-implemented method for supporting users in an operating room is provided. The method includes obtaining spatial information indicative of a plurality of spatial regions in the operating room, obtaining tracking information indicative of a tracked pose of a medical instrument in the operating room, and obtaining one or more predefined spatial constraints for one or more of the plurality of spatial regions. The method also includes determining whether the one or more predefined spatial constraints are met based on the spatial information and the tracking information, associating the medical instrument with one of the plurality of spatial regions based on whether the one or more predefined spatial constraints are met, and triggering visual feedback according to one or more feedback parameters of the spatial region associated with the medical instrument, wherein the one or more feedback parameters define a perspective of the navigation view.

### PRIORITY CLAIM

This application is a continuation-in-part of U.S. patent application Ser. No. 18/761,420, filed Jul. 2, 2024, which claims priority under 35 U.S.C. § 119 to European Patent Application No. 23183394.8, filed 4 Jul. 2023, the entire contents of which are hereby incorporated by reference.

### TECHNICAL FIELD

The present disclosure generally relates to a method for supporting users in an operating room by triggering feedback regarding one or more medical instruments. A related system, computer program and carrier are also disclosed herein.

### BACKGROUND

In many clinical scenarios, clinical personnel such as surgeons wish to be provided with feedback regarding tracked poses of medical instruments. For example, surgeons may wish to be informed on a current pose of a handheld drill relative to a patient's body, or on an alignment of a pedicle screw driver in relation to a medical instrument, such as a trocar, handled by a robot.

Some surgical navigation systems can provide a surgeon with a single navigation view visualizing a tracked pose of a handheld medical instrument relative to a patient's body. In case multiple medical instruments are used, tracked poses of the multiple medical instruments may be visualized at the same time in the single navigation view. Especially if the tracked medical instruments are located far apart from one another (e.g., at different vertebral levels of a patient's spine), or if multiple medical instruments are handled simultaneously by different surgeons, a single navigation view may not be optimal for navigating each of the medical instruments.

Some surgical navigation systems enable a user to select one of a plurality of tracked medical instruments and subsequently generate a single navigation view tailored to the selected medical instrument. This approach requires user interaction with the navigation system and only provides feedback for the selected medical instrument.

### SUMMARY

There is a need for a technique for supporting users in an operating room that solves one or more of the aforementioned or other problems.

According to a first aspect, a method for supporting users in an operating room by triggering feedback regarding one or more medical instruments is provided. The method is performed by at least one processor. The method comprises obtaining spatial information indicative of a plurality of spatial regions in the operating room, each of the plurality of spatial regions being associated with one or more feedback parameters. The method further comprises obtaining tracking information indicative of tracked poses of a plurality of medical instruments in the operating room. The method comprises associating, based on the spatial information and the tracking information, each of the plurality of medical instruments to a respective at least one of the plurality of spatial regions. The method comprises triggering feedback, for each of the plurality of medical instruments, according to the one or more feedback parameters of the associated respective at least one of the plurality of spatial regions.

The one or more feedback parameters may be region-specific. The one or more feedback parameters may differ between two or more (e.g., all) of the plurality of spatial regions.

The plurality of spatial regions may be defined based on a pose of at least one reference object in the operating room.

The at least one reference object may comprise at least a portion of a patient's body. The at least one reference object may comprise at least one anatomical element (e.g., a bone such as a vertebra) of the patient's body. The at least one reference object may comprise at least a portion of a medical instrument, for example a distal tool tip. The at least one reference object may comprise at least a portion of a medical instrument, for example a distal tip of the medical instrument.

In one example, different ones of the plurality of spatial regions comprise different portions of the patient's body.

At least some of the plurality of spatial regions may be separated from one another by one or more virtual planes. The virtual planes may be defined relative to the patient's body (e.g., relative to one or more anatomical elements of the patient's body).

The one or more virtual planes may comprise at least one anatomical plane, at least one user-defined plane and/or at least one plane associated with an anatomical element of the patient's body.

In one example, the medical instrument is handled by a robot in the operating room.

At least one of the spatial information and the tracking information may be obtained for multiple points in time. The feedback may be iteratively triggered for two or more (e.g., each) of the multiple points in time.

One or more of the associated respective at least one of the plurality of spatial regions may be updated based on a movement of one or more of the plurality of medical instruments as indicated by the tracking information obtained for multiple points in time.

The associated respective at least one of the plurality of spatial regions may be updated such that the medical instrument associated with said respective at least one of the plurality of spatial regions remains within said at least one of the plurality of spatial regions. Alternatively, or in addition, the associated respective at least one of the plurality of spatial regions may be updated such that a medical instrument not associated with said respective at least one of the plurality of spatial regions remains outside said at least one of the plurality of spatial regions.

In one variant, the plurality of medical instruments comprises instruments handled simultaneously.

The instruments handled simultaneously may be handled by different surgeons. In one example, different subsets of the plurality of spatial regions are

associated with different surgeons.

The one or more feedback parameters may define at least one of an auditory feedback, a haptic feedback and a visual feedback.

The visual feedback may include display of a navigation view visualizing the pose of the respective medical instrument.

In one example, the one or more feedback parameters define at least one setting of the navigation view, the at least one setting comprising: a type of medical image data used for rendering the navigation view; a criterion for highlighting structures in the navigation view; a criterion for indicating planned objects in the navigation view; an orientation of the navigation view; and/or a perspective of the navigation view.

According to a second aspect, a method for supporting users in an operating room by triggering visual feedback is provided. The method comprises obtaining spatial information indicative of a plurality of spatial regions in the operating room, obtaining tracking information indicative of a tracked pose of a medical instrument in the operating room, and obtaining one or more predefined spatial constraints for one or more of the plurality of spatial regions. The method further comprises determining, based on the spatial information and the tracking information, whether the one or more predefined spatial constraints are met. Then, based on whether the one or more predefined spatial constraints are met, the method further comprises associating the medical instrument with one of the plurality of spatial regions. Finally, the method comprises triggering visual feedback according to one or more feedback parameters of the spatial region associated with the medical instrument, the one or more feedback parameters defining a perspective of the navigation view.

The one or more predefined spatial constraints may comprise at least one of: a maximum distance between the instrument and the spatial region, a maximum distance between the instrument and an anatomical element enclosed by and/or defining the spatial region, a maximum deviation of a main instrument axis from the spatial region, and a maximum deviation of a main instrument axis from an anatomical element enclosed by and/or defining the spatial region. At least one of the spatial information and the tracking information may be obtained for multiple points in time and the visual feedback is iteratively triggered for two or more of the multiple points in time. The plurality of spatial regions may be separated from one another by one or more virtual planes defined relative to a patient. The spatial information may be determined or defined based on patient image data comprising one or more medical images of at least a portion of a patient's body.

According to a third aspect, a method for supporting users in an operating room by triggering visual feedback is provided. The method comprises obtaining a computer model representative of at least a portion of a patient in the operating room, obtaining planning data including a planned trajectory defined relative to a coordinate system associated with the computer model, obtaining spatial information indicative of a first spatial region associated with a first side of the patient and a second spatial region associated with a second side of the patient, and obtaining tracking information associated with a medical instrument. The method further comprises determining a current trajectory of the medical instrument relative to the coordinate system associated with the computer model based on the tracking information, as well as determining whether the medical instrument is approaching the patient from the first side or the second side based on the spatial information and tracking information. Finally, the method comprises controlling display of a navigation indicator visualizing a spatial relationship between the planned trajectory and the current trajectory based on whether the medical instrument is approaching the patient from the first side or the second side.

The method may further comprise causing the navigation indicator to: visualize a first direction of movement of the medical instrument as a first direction of movement of the current trajectory of the medical instrument relative to the planned trajectory in response to the medical instrument approaching the patient from the first side, and visualize the first direction of movement of the medical instrument as a second direction of movement of the current trajectory of the medical instrument relative to the planned trajectory in response to the medical instrument approaching the patient from the second side.

The planned trajectory may include a planned entry point and a planned target. The current trajectory may include at least one of a current position of a distal tip of the medical instrument and a current position of a shaft of the medical instrument.

The spatial information may be defined relative to the coordinate system associated with the computer model.

The navigation indicator may include crosshairs representative of the planned trajectory and a representation of the current position of the/a distal tip of the medical instrument. In such implementations, the method further may comprise: causing the representation of the current position of the distal tip of the medical instrument to move in a first direction relative to the crosshairs in response to the medical instrument being moved towards the planned trajectory and the medical instrument approaching the patient from the first side, and causing the representation of the current position of the distal tip of the medical instrument to move in a second direction relative to the crosshairs in response to the medical instrument being moved towards the planned trajectory and the medical instrument approaching the patient from the second side. Further, the navigation indicator may comprise a representation of the current position of the the/a shaft of the medical instrument. Here, the method may further comprise: causing the representation of the current position of the shaft of the medical instrument to move in a first direction relative to the representation of the current position of the distal tip of the medical instrument in response to the medical instrument being tilted towards an planned trajectory and the medical instrument approaching the patient from the first side, and causing the representation of the current position of the shaft of the medical instrument to move in a second direction relative to the representation of the current position of the distal tip of the medical instrument in response to the medical instrument being tilted towards the planned trajectory and the medical instrument approaching the patient from the second side.

The display of the navigation indicator may be controlled in response to the medical instrument being within a threshold distance of an anatomical target.

The tracking information may comprise a first position of the medical instrument corresponding to a first time and a second position of the medical instrument corresponding to a second time. In such implementations, the method may further comprise calculating an approach direction of the medical instrument based on the first and second positions of the medical instrument and determining whether the medical instrument is approaching the patient from the first side or the second side based on the calculated approach direction.

According to a fourth aspect, a method for supporting users in an operating room by displaying a representation of image data is provided. The method comprises obtaining image data representative of at least a portion of a patient in the operating room, obtaining spatial information indicative of a first spatial region associated with a first side of the patient in the operating room and a second spatial region associated with a second side of the patient in the operating room, and obtaining tracking information associated with a medical instrument in the operating room. The method further comprises determining whether the medical instrument is approaching the patient from the first side or the second side based on the spatial information and tracking information, and selecting (i) a first navigation view perspective in response to the medical instrument approaching the patient from the first side, or (ii) a second navigation view perspective in response to the medical instrument approaching the patient from the second side. Finally, the method comprises displaying a representation of the image data based on the selected navigation view perspective on a display.

The first navigation view perspective may be mirrored relative to the second navigation view perspective.

In some implementations, the method may further comprise overlaying a representation of the medical instrument over the representation of the image data based on the image data and the tracking information, and determining that the medical instrument is moving towards an anatomical target. In such implementations, the overlaid representation of the medical instrument may be displayed as moving towards the anatomical target and (i) in a first direction relative to the user when the first navigation view perspective is selected, or (ii) in a second direction relative to the user when the second navigation view perspective is selected.

The representation of the medical instrument may include a rendering of the medical instrument, and the rendering of the medical instrument may be overlaid over the representation of the image data based on the selected navigation view perspective. The rendering of the medical instrument may include a shape and an orientation. The rendering of the medical instrument may have the same shape when either of the first and second navigation view perspectives are selected. The orientation of the rendering of the medical instrument relative to the display may be changed based on the selected navigation view perspective. The orientation of the rendering of the medical instrument relative to the representation of the image data may be unchanged based on the selected navigation view perspective. The rendering of the medical instrument may be a three-dimensional rendering including a profile and a contour. The rendering of the medical instrument may have the same profile when either of the first and second navigation view perspectives are selected. The contour of the rendering of the medical instrument relative to the display may be changed based on selected navigation view perspective.

The navigation view perspective may be selected in response to the medical instrument being within a threshold distance of an anatomical target.

The tracking information may include a first position of the medical instrument corresponding to a first time and a second position of the medical instrument corresponding to a second time. In such implementations, the method may further comprise calculating an approach direction of the medical instrument based on the first and second positions of the medical instrument, and determining whether the medical instrument is approaching the patient from the first side or the second side based on the calculated approach direction.

According to a fifth aspect, a system is provided. The system comprises at least one processor configured to cause the system to perform the method according to any of the above aspects. For example, the system may include at least one processor configured to: obtain spatial information indicative of a plurality of spatial regions in the operating room, each of the plurality of spatial regions being associated with one or more feedback parameters; obtain tracking information indicative of tracked poses of a plurality of medical instruments in the operating room; associate, based on the spatial information and the tracking information, each of the plurality of medical instruments to a respective at least one of the plurality of spatial regions; and trigger feedback, for each of the plurality of medical instruments, according to the one or more feedback parameters of the associated respective at least one of the plurality of spatial regions. The at least one processor may be configured to perform the method according to the first aspect.

The system may further comprise a tracking system configured to track the poses of the plurality of medical instruments in the operating room. Alternatively, or in addition, the system may comprise a feedback unit configured to provide the feedback to at least one user in the operating room. The system may comprise a robot configured to handle a medical instrument.

According to a sixth aspect, a computer program is provided. The computer program comprises instructions which, when executed on at least one processor, cause the at least one processor to perform the method according to any of the above aspects. For example, with respect to the first aspect, the instructions, when executed by at least one processor, cause the at least one processor to: obtain spatial information indicative of a plurality of spatial regions in the operating room, each of the plurality of spatial regions being associated with one or more feedback parameters; obtain tracking information indicative of tracked poses of a plurality of medical instruments in the operating room; associate, based on the spatial information and the tracking information, each of the plurality of medical instruments to at least one of the plurality of spatial regions; and trigger feedback, for each of the plurality of medical instruments, according to the one or more feedback parameters of the associated at least one of the plurality of spatial regions. The computer program may comprise instructions which, when executed on the at least one processor, cause the at least one processor to perform the method according to the first aspect.

According to a seventh aspect, a carrier is provided. The carrier carries the computer program according to the third aspect. The carrier may carry a computer program comprising instructions which, when executed on the at least one processor, cause the at least one processor to perform the method according to any of the above aspects. For example, with respect to the first aspect, a carrier (e.g., a non-transitory computer storage medium) may be provided carrying (e.g., storing) a computer program comprising instructions which, when executed on at least one processor, cause the at least one processor to: obtain spatial information indicative of a plurality of spatial regions in the operating room, each of the plurality of spatial regions being associated with one or more feedback parameters; obtain tracking information indicative of tracked poses of a plurality of medical instruments in the operating room; associate, based on the spatial information and the tracking information, each of the plurality of medical instruments to at least one of the plurality of spatial regions; and trigger feedback, for each of the plurality of medical instruments, according to the one or more feedback parameters of the associated at least one of the plurality of spatial regions.

### BRIEF DESCRIPTION OF THE DRAWINGS

Advantages of the present disclosure will be readily appreciated as the same becomes better understood by reference to the following detailed description when considered in connection with the accompanying drawings.

FIG. 1 shows a schematic illustration of a system in accordance with the present disclosure.

FIG. 2 shows a flowchart of a method in accordance with the present disclosure.

FIGS. 3A-3F schematically illustrate spatial regions in accordance with the present disclosure.

FIGS. 4A-4F schematically illustrate different scenarios and corresponding spatial regions in accordance with the present disclosure.

FIG. 5 shows a perspective view of a computer model and a representation of a medical instrument.

FIG. 6 shows a navigation view including a navigation indicator.

FIG. 7A shows a perspective view of a surgical navigation system with a medical instrument in a first position and a navigation indicator set to a first perspective.

FIG. 7B shows a perspective view of the surgical navigation system of FIG. 7A with the medical instrument in a second position and the navigation indicator set to the first perspective.

FIG. 8A shows a perspective view of a surgical navigation system of FIG. 7A with the medical instrument in the first position and the navigation indicator set to a second perspective.

FIG. 8B shows a perspective view of the surgical navigation system of FIG. 7A with the medical instrument in the second position and the navigation indicator set to the second perspective.

FIG. 9A shows a perspective view of a surgical navigation system with a medical instrument in a first position and a navigation view set to a first perspective.

FIG. 9B shows the navigation view of FIG. 9A.

FIG. 10A shows a perspective view of a surgical navigation system with a medical instrument in a second position and a navigation view set to a second perspective.

FIG. 10B shows the navigation view of FIG. 10A.

### DETAILED DESCRIPTION

In the following description, exemplary embodiments will be explained with reference to the drawings. The same reference numerals will be used to denote the same or similar structural features.

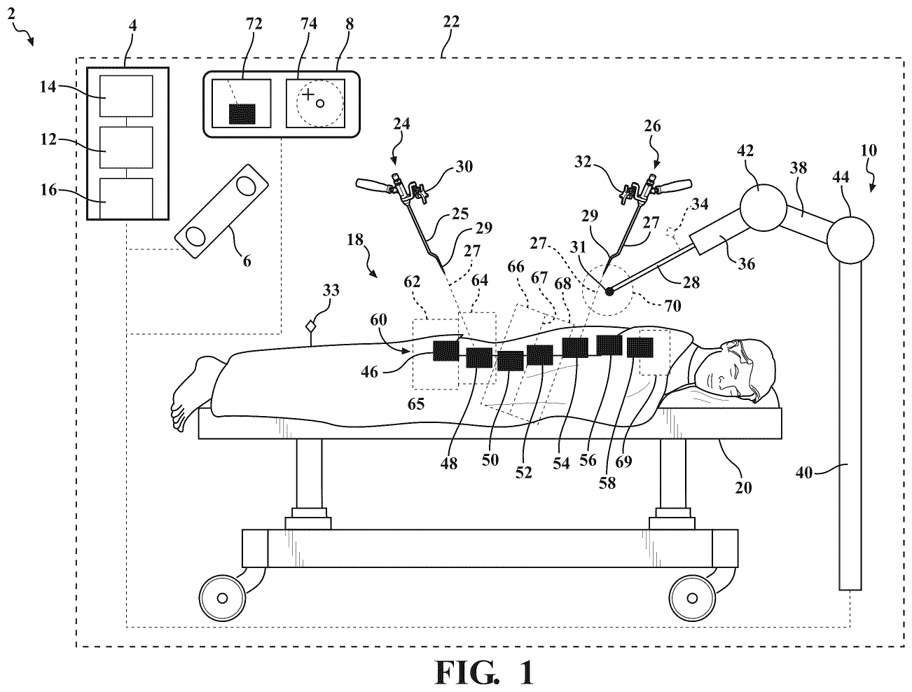

FIG. 1 shows a schematic illustration of a system 2 in accordance with the present disclosure. The system 2 may be referred to as a surgical navigation system and comprises a computing system 4. The system 2 may further comprise a tracking system 6, a display unit 8 and/or a robot 10. The computing system 4 comprises at least one processor 12 communicatively connected to at least one memory 14 and at least one interface 16. The at least one interface may be communicatively connected to the tracking system 6, the display unit 8 and/or the robot 10 via one or more wired or wireless connections.

Also shown is a patient's body 18 of a patient lying on a treatment bed 20 in an operating room 22, two medical instruments 24, 26 and a medical instrument 28. Each of the medical instruments 24, 26 in this example is a handheld instrument such as a pointer, a drill, a chisel or a screwdriver. The medical instruments 24, 26 may be handled simultaneously, for example by different surgeons. The medical instrument 24 comprises a shaft 25 extending longitudinally along a shaft axis 27. The medical instrument 26 comprises a distal instrument tip 29. The medical instrument 28 in the illustrated example is (e.g., simultaneously) handled by the robot 10 and comprises a distal tool tip 31.

The tracking system 6 is configured to track poses of the medical instruments 24, 26 by locating (e.g., optical or electromagnetic) trackers 30, 32 attached to the medical instruments 24, 26. The tracking system 6 may also track a pose of the medical instrument 28 by locating a tracker 34 attached thereto. The tracking system 6 may be configured as an optical tracking system and may comprise a (e.g., stereo-) tracking camera for locating optical trackers. The tracking system 6 may alternatively be configured as an electromagnetic tracking system and may comprise an electromagnetic field generator.

Alternatively to tracking the medical instrument 28 with the tracking system 6, a pose of the medical instrument 28 may be determined (e.g., by the at least one processor 12) based on a pose of the robot 10, for example based on rotations and/or translations of actuators of the robot 10. In the illustrated example, a pose of the medical instrument 28 may be determined based on angular orientations of arm segments 36, 38, 40 of the robot 10 defined by joints 42, 44 between the arm segments 36, 38, 40 of the robot 10.

The tracking system 6 may be further configured to track a pose of the patient's body 18 by locating a patient tracker 33 arranged in a fixed spatial relationship relative to the patient's body 18 (e.g., adhesively attached to the patient's skin or clamped to an anatomical element of the patient's body). A transformation, also known as a registration, between (e.g., pre-operative) patient image data of at least a portion of the patient's body 18 on the one hand and the patient's body 18 on the other hand may be used to transform (e.g., pre-planned) locations defined relative to the patient image data into (e.g., real-world) locations in the operating room 22. Various techniques for determining such a transformation or registration are known to those skilled in the art. For example, points acquired on a surface of the patient's body 18 using a tracked registration probe may be matched to a surface of the patient's body 18 as represented by the patient image data to obtain the transformation.

The display unit 8 is an example of a feedback unit configured to provide feedback to at least one user, in particular feedback regarding the medical instrument(s) 24, 26, 28. Other examples of such a feedback unit include a haptic feedback unit (e.g., included in one or more of the instruments 24, 26, 28), another type of visual feedback unit such as an indicator light (e.g., included in one or more of the instruments 24, 26, 28), and an auditory feedback unit such as a speaker.

The patient's body 18 comprises a plurality of anatomical elements such as organs or bones. In the illustrated example, vertebrae 46-58 of the patient's spine 60 are indicated as examples of anatomical elements of the patient's body 18. It is noted that although a patient's spine 60 typically comprises a total of 33 vertebrae, only seven vertebrae 46-58 are illustrated in FIG. 1 to provide a better overview. Also indicated in FIG. 1 are a plurality of spatial regions 60-68.

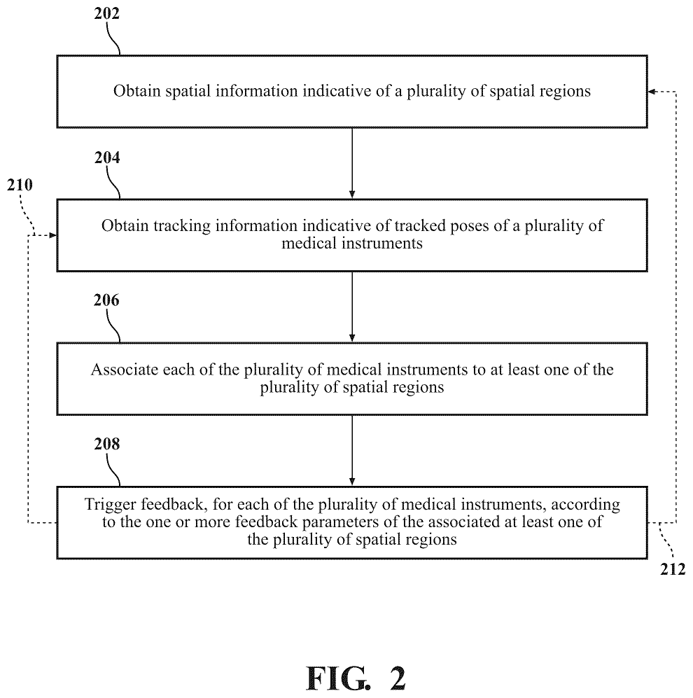

FIG. 2 shows a flowchart of a method in accordance with the present disclosure. The method may be performed by the at least one processor 12 of the system 2. To this end, the at least one memory 14 may store a computer program comprising instructions which, when executed by the at least one processor 12, cause the at least one processor to perform the method disclosed herein. The at least one memory 14 is an example of a carrier carrying the computer program. The computer program may alternatively be carried by a data stream received by the at least one processor 12 via the interface 16, for example from a server. Although reference signs of FIG. 1 will be used in the following, these are not to be understood as limiting the method to the specific details shown in FIG. 1.

The method may be referred to as a computer-implemented method. In one particular variant, the method is not a method for treatment of the human or animal body by surgery or therapy and/or the method does not comprise a surgical step.

In step 202, spatial information indicative of a plurality of spatial regions 62-70 in the operating room 22 is obtained.

The spatial information may indicate, for at least two or all of the spatial regions, at least one property selected from a size, a shape, an outline, a border, a position and an orientation. The spatial information may be obtained from the at least one memory 14. The spatial information may be predefined and/or pre-planned, in particular before a surgical procedure is started. The spatial information may be determined by the at least one processor 12 or defined by a user.

The spatial information may be determined or defined based on (e.g., pre-operative) patient image data comprising one or more medical images of at least a portion of the patient's body 18 such as computed tomography, CT, images and/or magnetic resonance, MR, images. The spatial information may be indicative of the spatial regions 62-70 in a real-world coordinate system, for example a coordinate system of the patient's body 18 and/or the patient tracker 33. The method may comprise obtaining (e.g., predefined and/or pre-planned) information indicative of the spatial regions 62-70 (e.g., defined based on or relative to one or more anatomical elements of the patient's body represented by the patient image data) in a coordinate system of the patient image data, obtaining a transformation between the coordinate system of the patient image data and a (e.g., the) real-world coordinate system, and determining the spatial information by transforming the information indicative of the spatial regions 62-70 from the coordinate system of the patient image data into the real-world coordinate system based on the obtained transformation. As explained above, various techniques exist for determining such a transformation, also referred to as registration between the patient image data and the patient's body 18.

One or more (e.g., all) of the plurality of spatial regions 62-70 may be defined (e.g., in the real-world coordinate system) based on a pose of at least one reference object in the operating room 22. For example, one or more (e.g., all) of the plurality of spatial regions 62-70 may be defined relative to the pose of the at least one reference object in the operating room 22 (e.g., in the real-world coordinate system). The at least one reference object may comprise at least a portion of the patient's body 18 (e.g., one or more anatomical elements of the patient's body 18 such as one of the vertebrae 46-58) and/or at least a portion of the medical instrument 28 (e.g., the distal tip 31 of the medical instrument 28) and/or at least a portion of a medical instrument 24, 26.

The plurality of spatial regions 62-70 may differ from one another in at least one property selected from a size, a shape, an outline, a border, a position and an orientation. The plurality of spatial regions 62-70 may differ from one another in one or more anatomical elements of the patient's body 18 comprised in the respective spatial region. In other words, different ones of the plurality of spatial regions 62-70 may comprise different portions of the patient's body (e.g., different anatomical elements such as different vertebrae 46-58). Each of the spatial regions 62-70 may be a three-dimensional region. Two or more (e.g., all) of the spatial regions 62-70 may be spatially disjunct. Two or more of the spatial regions 62-70 may overlap one another (e.g., at least partially).

At least some (e.g., two or more) of the plurality of spatial regions 62-70 may be separated from one another by one or more virtual planes 65, 37. The one or more virtual planes 65, 67 may be defined relative to the patient's body 18, for example relative to one or more anatomical elements (e.g., one or more vertebrae 46-58) of the patient's body 18. The one or more virtual planes 65, 67 may comprise at least one anatomical plane (e.g., a coronal plane, a sagittal plane or a transverse plane), at least one user-defined plane and/or at least one plane associated with an anatomical element of the patient's body 18 (e.g., one of the vertebrae 46-58). The method may comprise obtaining information (e.g., from a DICOM header of the patient image data) indicative of an orientation of the at least one virtual plane 65, 67 in the coordinate system of the patient image data and transforming the orientation into the real-world coordinate system, using the known registration between these two coordinate systems, to obtain the two or more spatial regions 62-70, which separated by the at least one virtual plane 65, 67, in the real-world coordinate system.

Different subsets of the plurality of spatial regions may be associated with different surgeons. That is, different spatial regions may be defined for different surgeons pre-operatively. Each of the subsets of the plurality of spatial regions may correspond to a different side of the patient's spine 60, for example relative to an anatomical plane. The subsets may consist of subset-specific spatial regions. That is, none of the plurality of spatial regions may be associated with more than one surgeon.

Each of the plurality of spatial regions 62-70 is associated with one or more feedback parameters.

The one or more feedback parameters may be predefined and/or obtained from the at least one memory 14. The one or more feedback parameters may be defined by a user. The one or more feedback parameters may differ between two or more of the plurality of spatial regions. In one example, the one or more feedback parameters are region-specific. The one or more feedback parameters may be specific for anatomical elements of the patient's body comprised in the respective spatial regions. This may enable a feedback that is tailored specifically for the individual spatial zones.

In step 204, tracking information indicative of tracked poses of a plurality of medical instruments in the operating room is obtained (e.g., tracked poses of the instruments 24, 26, 28 in the real-world coordinate system).

The tracking information may be obtained from the tracking system 6 by the at least one processor 12, for example via the at least one interface 16. Alternatively, the tracking information may be loaded from the at least one memory 14. The tracking information may be indicative of exactly one pose per medical instrument 24, 26, 28, for example tracked at a same point in time. Thus, the tracking information may indicate poses of the tracked medical instruments 24, 26, 28 relative to one another.

The order of steps 202 and 204 may be reversed compared with FIG. 2. In another variant, the spatial information and the tracking information are obtained simultaneously (e.g., as part of a single dataset) such that steps 202 and 204 are performed in unison.

In step 206, each of the plurality of medical instruments (e.g., 24, 26, 28) is associated to a respective at least one of the plurality of spatial regions (e.g., 62-70), based on the spatial information and the tracking information.

Each of the medical instruments 24, 26, 28 may be associated to a subset of the spatial regions 62-70, or to exactly one of the spatial regions 62-70. For example, a medical instrument may be associated to a spatial region 62-70 in case one or more predefined spatial constraints of this spatial region 62-40 are met. The one or more predefined spatial constraints may be obtained from the at least one memory 14. The method (e.g., step 206) may comprise obtaining the one or more predefined spatial constraints for one or more of the plurality of spatial regions 62-70 and determining, based on the spatial data and the tracking data, whether the one or more predefined spatial constraints are met. The one or more predefined spatial constraints may comprise at least one of (i) a maximum distance between (e.g., at least one part of) the instrument (e.g., the instrument's distal tip) and the spatial region, (ii) a maximum distance between (e.g., at least one part of) the instrument (e.g., the instrument's distal tip) and an anatomical element enclosed by and/or defining the spatial region, (iii) a maximum deviation of a main instrument axis (e.g., a longitudinal shaft axis 27) from the spatial region and (iv) a maximum deviation of a main instrument axis (e.g., a longitudinal shaft axis 27) from an anatomical element enclosed by and/or defining the spatial region. A spatial region closest to the medical instrument and/or a spatial region pointed at by the medical instrument may be associated with that medical instrument or vice versa.

In the example of FIG. 1, the medical instrument 24 may be associated to the spatial region 64, as the longitudinal shaft axis 27 intersects the spatial region 64 which means the predefined spatial constraint of a maximum deviation of the longitudinal shaft axis 27 from the spatial region 64 is met. The medical instrument 26 may be associated to the spatial region 70, as the distal tip 29 lies within the spatial region 70 which means the predefined spatial constraint of a maximum distance of the distal tip 29 from the spatial region 70 is met.

In one variant, predefined association information is obtained indicative of associations of each of the plurality of medical instruments (e.g., 24, 26, 28) to a respective at least one of the plurality of spatial regions (e.g., 62-70). The associations may be defined by a user. The associations may be determined based on a priority associated with each of the plurality of medical instruments.

In step 208, for each of the plurality of medical instruments, feedback is triggered, according to the one or more feedback parameters of the associated respective at least one of the plurality of spatial regions.

The triggered feedback may be based on at least one of the tracking information and the spatial information. The feedback triggered for a medical instrument may be based on the tracked pose of this medical instrument (e.g., as indicated by the tracking information) and based on the associated (e.g., respective) at least one of the plurality of spatial regions. The feedback triggered for a medical instrument may be indicative of the tracked pose of this medical instrument, for example relative to the associated (e.g., respective) at least one of the plurality of spatial regions. The one or more feedback parameters associated with a spatial region may define how the tracked pose of the medical instrument associated with this spatial region is indicated by the feedback triggered for this medical instrument.

The one or more feedback parameters may define at least one of an auditory feedback (e.g., a warning tone), a haptic feedback (e.g., a vibration) and a visual feedback (e.g., display of an image on the display unit 8 or activation of a warning lamp). The visual feedback may include display of a navigation view 72, 74 visualizing the pose of the respective medical instrument 24, 26, 28 as indicated by the tracking information. In this case, the one or more feedback parameters may define at least one setting of the navigation view 72, 74, the at least one setting comprising: a type of medical image data used for rendering the navigation view 72, 74; a criterion for highlighting structures in the navigation view 72, 74; a criterion for indicating planned objects in the navigation view 72, 74; an orientation of the navigation view 72, 74; and/or a perspective of the navigation view 72, 74.

In the example of FIG. 1, the navigation view 72 may indicate a pose of the medical instrument 24 by visualizing the longitudinal shaft axis 27 relative to the anatomical element encased by the spatial region 64, namely the vertebra 48. In this example, the one or more feedback parameters associated with the spatial region 64 may define a visual feedback including display of the navigation view 72, and may define a perspective of the navigation view as a lateral perspective. The navigation view 74 may indicate a pose of the medical instrument 26 by visualizing the location of the tip 29 in the form of crosshairs relative to the location of the tool tip 31, and by further visualizing the spatial region 70. In this example, the one or more feedback parameters associated with the spatial region 70 may define a visual feedback including display of the navigation view 74, and may define a perspective of the navigation view as a perspective along a main axis of the instrument's 26 shaft.

At least one of the spatial information and the tracking information may be obtained for multiple points in time and the feedback may be iteratively triggered for two or more (e.g., each) of the multiple points in time. The method may be at least partially repeated by updating at least one of the spatial information and the tracking information and performing the subsequent steps 206-208 using the updated information, as indicated in FIG. 2 with dashed arrows 210, 212. This may ensure that the navigation view 72, 74 indicates a current pose of a medical instrument 24, 26, 28 and may thus be particularly advantageous for guiding a user.

One or more of the associated respective at least one of the plurality of spatial regions 62-70 may be updated based on a movement of one or more of the plurality of medical instruments 24, 26, 28 as indicated by the tracking information obtained for multiple points in time. For example, the associated respective at least one of the plurality of spatial regions (e.g., 70) may be updated such that the medical instrument (e.g., 26) associated with said respective at least one of the plurality of spatial regions remains within said at least one of the plurality of spatial regions. Alternatively, or in addition, the associated respective at least one of the plurality of spatial regions (e.g., 70) may be updated such that a medical instrument (e.g., 24) not associated with said respective at least one of the plurality of spatial regions remains outside said at least one of the plurality of spatial regions. Thus, spatial zones may be updated based on a movement of the medical instruments 24, 26, 28 associated or not associated with these spatial zones. This may ensure that the association between a spatial zone and a medical instrument is maintained over time.

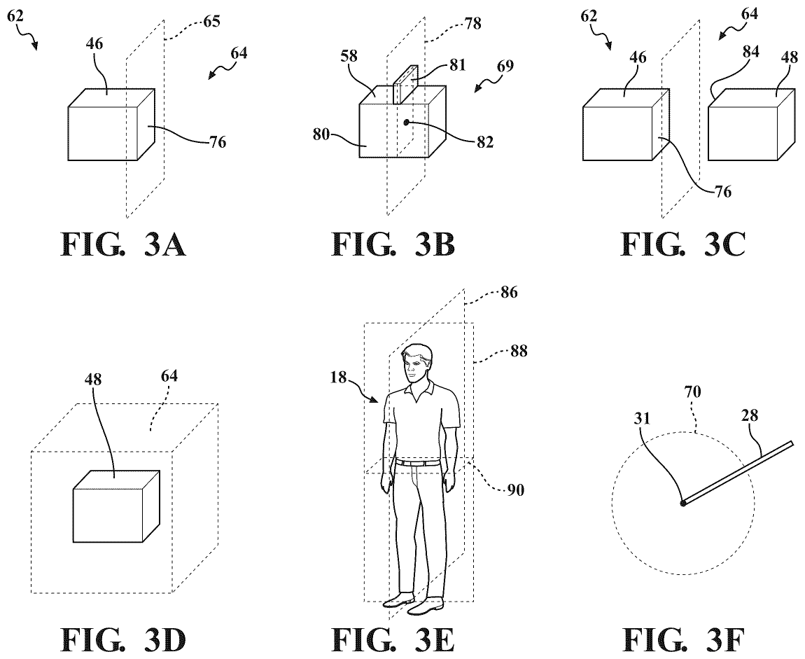

FIGS. 3A-3F schematically illustrate examples of spatial regions in accordance with the present disclosure.

In the example of FIG. 3A, a virtual plane 65 defines a boundary between two adjacent spatial regions 62, 64. In this example, the virtual plane 65 is defined based on an anatomical element of the patient's body 18, namely a vertebra 46 of the patient's spine 60. A surface of the anatomical element, namely an endplate 76 of the vertebra 46, lies in the virtual plane 65.

In the example of FIG. 3B, a virtual plane 78 defines a border of a spatial region 69. In this example, the virtual plane 78 is defined relative to an anatomical element of the patient's body 18, namely a vertebra 58 of the patient's spine 60. The virtual plane 78 extends axially along a middle of the spinous process 81 of the vertebra 58. The virtual plane 78 may be perpendicular to the endplate 80 of the vertebra 58, and intersect a center 82 of the vertebra 58.

In the example of FIG. 3C, a virtual plane 65 defines a boundary between two adjacent spatial regions 62, 64. In this example, the virtual plane 65 is defined based on two anatomical elements of the patient's body 18, namely two vertebrae 46, 48 of the patient's spine 60. The virtual plane 65 extends (e.g. equidistantly) between the two anatomical elements and in the illustrated example is parallel to an endplate 76, 84 of each vertebra 46, 48.

In the example of FIG. 3D, a spatial region 64 is defined as a bounding box around an anatomical element of the patient's body 18, for example a vertebra 48. The bounding box may have a predefined shape such as a square or a sphere, or may define an area with a predefined thickness around the enclosed anatomical element, thereby representing an enlarged version of the anatomical element.

In the example of FIG. 3E, a plurality of virtual planes 86, 88, 90 are illustrated. The virtual planes 86, 88, 90 correspond to different anatomical planes of the patient's body 18. Each of the virtual planes 86, 88, 90 may be a border of one or more spatial regions and/or may separate adjacent regions from one another. In the example of FIG. 3E, a maximum of 8 spatial regions may be defined by the virtual planes 86, 88, 90. The virtual planes 86, 88, 90 may be defined relative to the coordinate system of the image data. The virtual planes 86, 88, 90 may also be defined based on segmented image data. For example, image data including vertebrae of the patient may be segmented, and at least one of the virtual planes 86, 88, 90 may be defined based on the segmented image data. In this example, the virtual plane 86 may be defined based on a line that passes through at least some of the segmented vertebrae. Further, since the coordinate system of the image data may be registered to the real world coordinate system, the pose of the virtual planes 86, 88, 90 relative to the rest of the operating room may be determined by the computing system 4.

In the example of FIG. 3F, a spherical spatial region 70 is illustrated, having a predefined radius (e.g., several cm) around a tool tip 31 of the medical instrument 28.

Other variants of spatial regions are also possible. For instance, a spatial region may be defined by a user (e.g., in an arbitrary pose relative to the patient's body). One or more spatial regions may be determined based on a segmentation of at least one anatomical element encased by the one or more spatial regions, such as in the example of FIG. 3D.

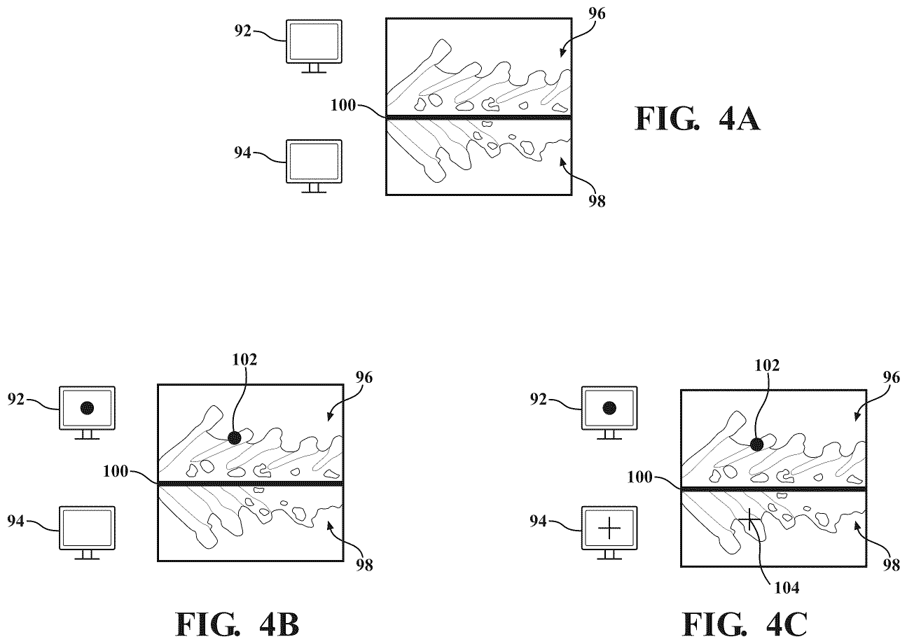

FIGS. 4A-4F schematically illustrate different scenarios and corresponding spatial regions in accordance with the present disclosure.

FIG. 4A shows two navigation views 92, 94 that may be displayed on a same display unit 8 or on different display units 8. A medical image of the patient's body 18 is also shown in FIG. 4A, on which spatial regions 96, 98 are indicated that are separated from one another by a virtual plane 100. In this example, the virtual plane 100 defining the spatial regions 96, 98 may correspond to the sagittal plane of the patient's body 18. Thus, the virtual plane 100 is static and the spatial zones 96, 98 shown in FIG. 4A may be referred to as static or initial spatial zones. One surgeon may operate in the spatial zone 96 and another surgeon may operate in the spatial zone 98. As no tracked instruments are visible to the tracking system 6 in the scenario of FIG. 4A, no such instruments are visualized in the navigation views 92, 94.

In the scenario of FIG. 4B, a surgeon has positioned a tip of a medical instrument (e.g., 26) at a position 102 relative to the patient's body 18. The tracking system 6 allows determining the relative pose between that medical instrument and the spatial zones 96, 98. The tracked instrument is associated to the spatial zone 96 in which it is located, and a corresponding navigation view 92 is displayed, indicating the pose of that medical instrument relative to the patient's body 18.

In the scenario of FIG. 4C, another surgeon has positioned a tip of another medical instrument (e.g., 26) at a position 104 relative to the patient's body 18. The tracking system 6 allows determining the relative pose between that medical instrument and the spatial zones 96, 98. The instrument at position 104 is associated to the spatial zone 98 in which its tip is located, and a corresponding navigation view 94 is displayed, indicating the pose of that medical instrument relative to the patient's body 18. As can be seen in FIG. 4C, the navigation view 92 is displayed simultaneously. This allows navigating both instruments relative to the patient's body 18 at the same time by different surgeons operating on the different spatial zones.

FIG. 4D illustrates a scenario in which the instrument associated with the spatial zone 96 is moved relative to the patient's body 18 from position 102 to position 108. Such a movement of the instrument may effect or trigger an update of the initial spatial region 96 and/or 98. In the illustrated example, the initial spatial zones 96, 98 are updated by using a virtual plane 106 instead of the virtual plane 100. The virtual plane 106 is perpendicular to a linear line connecting the positions 104 and 108 and intersects the linear line in its middle. This ensures that each of the positions 104, 108 has a similar distance to the edge of the respective spatial zone 96, 98. The association between the instruments and the updated spatial zones 96, 98 can be maintained and the navigation views can both be continually displayed in this scenario.

FIG. 4E illustrates a scenario in which the instrument associated with the spatial zone 96 is moved relative to the patient's body 18 from position 102 to position 110. Position 110 lies within the initial spatial zone 98 defined based on the virtual plane 100 as shown in FIG. 4A. To avoid the instrument from moving from between positions 102, 110 from the spatial zone 96 to the spatial zone 98, the initial spatial zones 96, 98 may be updated based on the instrument's movement. Again, the updated spatial zones 96, 98 may be defined by a virtual plane 109 perpendicular to a linear line connecting the positions 104 and 110 and intersecting that line in its middle. Also in this case, the association between the instruments and the spatial zones 96, 98 can be maintained and the navigation views 92, 94 can both be continually displayed.

In the scenario of FIG. 4F, the medical instrument previously located at position 104 is removed from the spatial region 98 and no longer within a tracking range of interest of the tracking system 6. Thus, the navigation view 94 may be left blank or stopped to be displayed. The navigation view 92 that is based on the instrument located at position 108 may not be affected by the removal of the instrument previously located at position 104. That is, the navigation view 92 may be continually displayed irrespective of a presence or absence of other medical instruments, as long as the medical instrument associated with the spatial zone 96 is within the tracking range of interest of the tracking system 6.

If, starting with the scenario of FIG. 4F, another medical instrument is positioned relative to the patient's body 18, that medical instrument may be associated with the (e.g., initial or updated) spatial region 98 if it is located within that region. This may lead to the same situation as shown in FIG. 4E. The initial spatial zones defined by the virtual plane 100 may be used as fixed zones used for associating a medical instrument to one of the spatial regions, and the spatial regions may subsequently be (e.g., continuously) updated based on (e.g., changed) tracked poses of the associated medical instruments.

Details of the inventive technique will now be phrased in other words to provide a better understanding thereof. Although reference signs of FIGS. 1-4F may be used in the following, these are not to be understood as limiting the inventive technique to the specific details shown in these figures.

Complications (e.g. surgical site infections or blood loss) during a surgery may increase with the duration of the surgical intervention. More than one surgeon performing a navigated surgery simultaneously can decrease the surgical time and therefore the overall complication rate. Allowing navigation of multiple medical instruments 24, 26, 28 simultaneously, so that each surgeon can operate on different vertebral levels or spinal sections at a time, can benefit in terms of reducing surgery time, reducing blood loss, and collaborating on a complex case. For example, in a scoliosis case where the spine 60 has a side-to-side bend which is estimated at 10 degrees or more, there may be a need for a spinal fusion. The basic motivation is to realign and fuse together some of the vertebrae 46-58 of the patient's spine 60 with additional support of rods, so that they heal into a single, solid bone. In such an operation, more than one surgeon may be operating on the patient's body 18 at a time. For example, one surgeon may implant pedicle screws on the patient's right side and another surgeon on the patient's left side of vertebral column. This may decrease turnaround time and reduce blood loss.

Some 3D navigation systems do not support simultaneous visualization of multiple navigated medical instruments. Only a view of a single medical instrument may be provided by such navigation systems. For example, in some systems, views are determined to be displayed (e.g., an axial view, a sagittal view, a coronal view, a view along a tool axis) using a tracked position of a distal tip 29 of the single medical instrument 26 as a reference for these views. If another medical instrument 24 is brought into a surgical region and is located closer to the patient's body 18 than the single instrument 26, then the single instrument 26 may no longer be considered as the primary medical instrument and the other medical instrument 24 may become the new primary medical instrument to be used for determining the view(s). In these cases, only one surgeon may navigate on the patient anatomy at a given time.

Some 2D navigation systems may visualize a view indicating multiple medical instruments 24, 26, 28 at the same time. For example, a static view of a portion of the patient's body 18 (e.g., an anterior-posterior, AP, and a lateral x-ray image) is shown on a display unit. A navigated medical instrument 24 is displayed as an overlay on both x-ray images. In this case, multiple instruments 24, 26, 28 can be overlaid in the x-ray images simultaneously. If the position of the medical instrument 24 effects a change in the view by changing a section of the x-ray images to be displayed as part of the view (e.g., in case the view corresponds to a zoomed version of an x-ray image close to the instrument's distal tip), a separate view for each navigated instrument 24, 26, 28 may need to be provided. In 3D navigation systems, intersecting 2D planes of 3D patient image data may be shown on a display unit. The intersecting planes may be derived from the tracked position of the distal tip 29 of the medical instrument 26. In such a view, for example, a sagittal and/or a coronal intersecting plane may be shown which includes the instrument tip 29. The sagittal plane may be parallel to a y-axis and a z-axis, and the coronal plane may be parallel to an x-axis and a y-axis of a DICOM coordinate system of the 3D patient image data. In another view, also referred to as instrument-oriented view, the instrument tip 29 may be shown overlaid onto an intersecting plane, but the intersecting plane may in this case be defined by a coordinate system of the instrument tip 29 instead of the 3D patient image data. In this example, one could say that the instrument tip 29 scrolls through the 3D data set. In cases where the instrument tip 29 controls the displayed view, it may be desirable to display different views for each of a plurality of simultaneously used medical instruments 24, 26, 28.

In navigated spinal surgery there might be a need to operate on several spinal levels or sections of a patient's spine 60 simultaneously by two or more surgeons. The technique disclosed herein allows providing simultaneous feedback for multiple tracked medical instruments 24, 26, 28 associated with surgeon-specific spatial regions, for example by providing a separate navigation view 72, 74 for each of the instruments 24, 26, 28. This may allow navigating multiple medical instruments 24, 26, 28 simultaneously, so that each surgeon can operate on a different spinal level or section of the spine 60 of the patient's body 18 at a given time. For example, one surgeon may perform a spinal decompression on a right side of the patient's spine 60 while another can implant a pedicle screw on left side thereof, or the two surgeons may implant pedicle screws on the respective sides of the vertebral column of the patient's spine 60. The navigation views 72, 74 may be displayed on a single screen of a display unit 8 as illustrated in FIG. 1, or on individual screens thereof.

As another example for visual feedback, a blinking pattern and/or light intensity of an indicator lamp may indicate a distance and/or an alignment of the tracked medical instrument 26 relative to the associated spatial region 70. Alternatively, or in addition to providing a visual feedback such as a navigation view or a light signal of the indicator lamp as feedback on the tracked poses of the multiple medical instruments 24, 26, 28, haptic and/or auditory feedback may be output to the user(s) in the operating room 22. For example, a vibration pattern and/or intensity of a haptic feedback unit worn by a surgeon may indicate a distance of the tracked medical instrument 24 relative to the associated spatial region 64. A warning tone may be output via a speaker to indicate that the distal tip 29 of the tracked medical instrument 26 is currently located outside the associated spatial region 70.

A surgeon may wish to sequentially use multiple tracked medical instruments during a procedure. The medical instrument that is actively used by a surgeon for surgical navigation and shall be used for providing feedback to the surgeon may be referred to as primary instrument 24, 26, 28. The presented technique allows providing feedback for multiple primary medical instruments 24, 26, 28 handled simultaneously, for example by displaying navigation views 72, 74 for the multiple primary medical instruments 24, 26, 28 while other medical instruments may or may not be visualized in these navigation views.

A primary medical instrument 24, 26, 28 may be selected from a plurality of tracked medical instruments by a user, for example by pressing a button on the primary medical instrument 24, 26, 28 or on a Graphical User Interface. Alternatively, a primary medical instrument 24, 26, 28 may be determined based on the tracking data and the spatial data, for example in case the tracked pose of the primary instrument 24, 26, 28 fulfils the one or more predefined spatial constraints. In one particular example, a medical instrument 26 can be selected as primary medical instrument if its distal tip 29 is located in the associated spatial region 70. If more than one medical instrument lies in a same spatial region 62-70, other criteria may be used for selecting the primary instrument from the more than one medical instrument, for example a tool located closest to the patient tracker 33 or closest to a center of the same spatial region 62-70 may be selected as the primary medical instrument. As another option, each medical instrument may have an associated priority that may be predefined (e.g., based on a type of the medical instrument) or based on the tracking information (e.g., based on a movement direction or movement speed of the medical instrument), and the primary instrument may be selected from multiple tracked medical instruments based on the priorities of these medical instruments.

The present technique may enable simultaneous navigation of multiple medical instruments 24, 26, 28, for example by providing corresponding navigation views 72, 74 to surgeons. One concept lies in providing a plurality of spatial regions 62-70. One primary medical instrument may be associated to each spatial region 62-70 or to a subset of the spatial regions 62-70. The patient's body 18 may be divided into multiple surgical zones as spatial regions 62-70 that are operated by different surgeons, for example by using separating virtual planes 65, 67, 78, 68, 88, 90 defining the spatial regions 62-70. The spatial regions 62-70 may be defined based on one or more of the following approaches (A) to (D).

### (A) Virtual Plane Based on Anatomical Plane

A sagittal plane derived from the coordinate system of the patient image data (e.g., a DICOM image dataset) may be used as a virtual plane to divide the patient's body 18 into a left and a right region. Several planes could be combined to create more spatial regions. For example, an additional transverse plane may be added to divide the left and right regions into cranial and a caudal sub-regions, resulting in a total of four spatial regions. Instead of using the anatomical planes as the virtual planes for defining the spatial regions, one may use planes that are parallel to the anatomical planes and which, for example, contain a (e.g., predefined) point of interest. Examples of such virtual planes 86, 88, 90 corresponding to or parallel to the anatomical planes are shown in FIG. 3D.

### (B) Virtual Plane Based on Anatomical Element(s)

The orientation of a virtual plane does not necessarily need to correspond to an anatomical axis as defined by the coordinate system of the patient image data. In another variant, the orientation of a virtual plane is defined relative to an anatomical element of the patient's body 18, such as a bone (e.g., a vertebra 46-58), an organ or a tumor. The orientation of the virtual plane may be derived from a segmentation of this anatomical element. One or more of the virtual planes may be determined based on a segmentation of the patient's spine 60. As an example, a virtual plane may approximate an endplate of a vertebra of the patient's body, as exemplarily illustrated in FIG. 3A. In this case, one surgeon may operate on superior levels relative to this virtual plane and another surgeon may operate on inferior levels in respect to this virtual plane. Several virtual planes could be defined in a similar manner, thereby dividing the patient's spine 60 into different spatial regions. Another example of a virtual plane defined based on an anatomical element of the patient's body is a virtual plane that approximates the spinous process of a vertebra and divides the patient's body 18 into a left and a right side, as exemplarily illustrated in FIG. 3B.

### (C) Virtual Plane Defined by a User

A user may define a spatial region and/or one or more of the virtual planes. For example, the one or more virtual planes can be defined by the user via a Graphical User Interface, GUI, based on the patient image data. In one example, the user defines two points in the patient image data to define an axis, and defines a point on this axis. A virtual plane may then be determined having the user-defined axis as normal and the user-defined point as intersection point with the user-defined axis. As another example, the user may draw freeform shapes in a plurality of views of the patient's body 18 as represented by the patient image data, and a spatial region may be determined which has the respective freeform shapes as contour. Other variants for defining virtual planes or spatial regions by a user are also possible.

### (D) General Geometric Object

The discriminating geometric object used to define a spatial region is not restricted to be a virtual plane. A general geometric (e.g., virtual) object such as a mesh-based surface model may be used to define one or more of the spatial regions. For example, an anatomical element in the patient image data may be segmented and a convex hull may be defined around the segmented anatomical element as a spatial region, as exemplarily illustrated in FIG. 3D. This spatial region may thus have a sub volume of the patient's body 18 as an interior or inside volume, and may divide the patient's body 18, and the patient image data representative of the patient's body 18, into an inside volume and outside volume.

In all cases (A) to (D), spatial regions can further be restricted by a bounding volume, for example a bounding box around the patient's body 18 and/or the patient image data.

When dividing the anatomy into n different spatial regions, the number of primary medical instruments may range from zero to n. A medical instrument 26, the distal tip 29 of which lies in one of the defined spatial regions, may be associated with this one of the spatial regions and thus become the primary medical instrument for this associated one of the spatial regions.

A navigation view 74 may be provided as visual feedback related to this primary medical instrument 26, the navigation view indicating a pose of the primary instrument 26 relative to the portion of the patient's body 18 as represented by the patient image data, the portion being contained in the spatial region 70 associated to the primary instrument 24. Each spatial region 62-70 can be navigated simultaneously and independently using a respective primary instrument 24, 26, 28. During navigation, spatial regions 64, 70 associated with the primary instruments 24, 26, 28 may be visualized concurrently on a single screen (e.g., as illustrated in FIG. 1) or on multiple screens. Multiple navigation views 72, 74 can be displayed on different windows of a same display unit 8 or on different display units. Each primary instrument 24, 26, 28 may be visualized in its own navigation view(s) 72, 74. In an anatomical navigation view this may for example be a 2D sagittal and 2D coronal view which contains the tip of the primary instrument. This may enable surgeons to collaborate and operate together.

Each of the tracked medical instruments may be classified (e.g., by the tracking system) into a surgeon-specific set of medical instruments. Different surgeons may use different instrument sets. These sets may be predefined or defined by a user (e.g., the surgeon(s)). In one example, one instrument per instrument set is used as a primary instrument. This means that only one instrument per instrument set may be associated to a spatial region 62-70 or a subset of spatial regions 62-70, and feedback may be triggered using the one or more feedback parameters of the spatial region(s) 62-70 associated with that primary instrument.

As explained above, one concept lies in dividing the patient's anatomy into two or more spatial regions 62-70 and associating one primary navigated medical instrument 24, 26, 28 to a spatial region. A fixed association between a medical instrument 24, 26, 28 and a spatial region 62-70 can be used. The displayed navigation view indicating a pose of the associated medical instrument 24, 26, 28 may be determined for the respective associated spatial region 62-70 to provide each surgeon with a dedicated visualization (e.g., on a surgeon-specific display unit). In contrast to such a region-based approach for identifying which portion of the patient's body to display in the navigation view, a direct association of a medical instrument to a display unit would not make it possible to visualize the same medical instrument on different display units for the surgeons without processing additional information like tracking of the surgeons to identify which instrument belongs to whom or adjusting manually for each display unit which tool should be used for navigation.

If a distal tip of a medical instrument of a surgeon A enters a spatial region defined for another surgeon B, there may no longer be a primary medical instrument in the spatial region for surgeon A, but two medical instruments may be simultaneously located in the spatial region for surgeon B. Only one medical instrument may be the primary one that controls the navigation view. Thus, in this scenario, one surgeon might “lose” his navigation view. For example, surgeon B may no longer be provided with a navigation view. This means A is taking over the control, where B does not see his instrument visualized anymore. To avoid this situation, the spatial regions may be dynamically updated once multiple surgeons are navigating simultaneously. The segregation between the spatial regions may be based on the current position of the tips of the tracked medical instruments. That is, static regions (e.g., defined relative to the patient's body) may be used initially and be updated subsequently based on a movement of the tracked medical instruments, in particular to avoid a tracked medical instrument from moving into another spatial region. One may say that the static regions can be employed as triggers to visualize a medical instrument on a specific display unit for a surgeon.

Once multiple surgeons are navigating the tracked medical instruments simultaneously and the navigation views are displayed (e.g., on dedicated display units for each surgeon), the respective associated spatial regions may be anchored to the distal tips of the tracked medical instruments. In other words, the segregation of the situs may be derived by the positions of the medical instruments.

For example, a virtual plane separating two adjacent spatial regions from one another may be a defined as a plane that is perpendicular to a line connecting the distal tips of two tracked (e.g., primary) medical instruments, which plane has the same distance to both distal tips. This concept of dividing the space into spatial regions is known from Voronoi diagrams. That is, each of the (e.g., updated) plurality of spatial regions may correspond to a Voronoi cell defined based on a point (e.g., a distal tip) of a tracked medical instrument. In a Voronoi diagram for a given set of points {p1, . . . , pn} the space is divided into so called Voronoi cells. The Voronoi cell for the point pi consists of every point whose distance to pi is less than its distance to any other point of the given point set. So, for n surgeons, n segregating virtual planes may be defined, thereby defining n Voronoi cells, one for each of the instrument tips pi, i=1, . . . , n. With this concept of dynamically dividing the space into Voronoi cells for each instrument tip, it is possible to provide a fixed navigation view for each surgeon, even if the tip of the primary instrument associated to that surgeon enters a spatial region that was initially provided for another surgeon.

If multiple surgeons are operating and one surgeon removes a medical instrument from the situs, the associated navigation view may be let blank while the others still show their navigated instrument tips. If the surgeon picks another medical instrument and inserts the instrument's tip into the spatial region defined for that surgeon, this instrument may again be visualized in the navigation view previously left blank. If there is already at least one instrument in that spatial region, a separation (e.g., a subdivision of that spatial region) may be performed with the new instrument and other instrument(s) in said region. The new instrument's tip may be shown on the display unit associated to the surgeon and all other instruments may still be visualized in their navigation view and/or on their display units.

Various modifications of the technique disclosed herein are possible. For example, technique presented herein is not restricted to spinal surgery and applies to other navigated surgical procedures (e.g., hip, knee, cranial or shoulder surgery).

The technique may allow more than one surgeon to navigate his primary medical instrument 24, 26, 28 simultaneously during a surgical navigation procedure. Navigating with multiple medical instruments 24, 26, 28 at the same time decreases the intervention duration and increases efficiency and effectivity of the intervention. When providing the feedback based on the spatial region associated with a tracked instrument, instead of providing a same feedback for all tracked instruments, a user may distinguish the feedback between the spatial regions 62-70, thereby correlating the respective primary instrument with the associated spatial region more easily. Region-specific feedback parameters may enable a feedback that is specifically tailored to the respective spatial region, which may be particularly advantageous compared with solutions in which the feedback on a tracked instrument pose is the same irrespective of the instrument's pose relative to a spatial region. When the spatial regions are associated to different surgeons, the one or more feedback parameters may be tailored to the needs and preferences of the different surgeons. Further advantages may become apparent to those skilled in the art in view of the present disclosure.

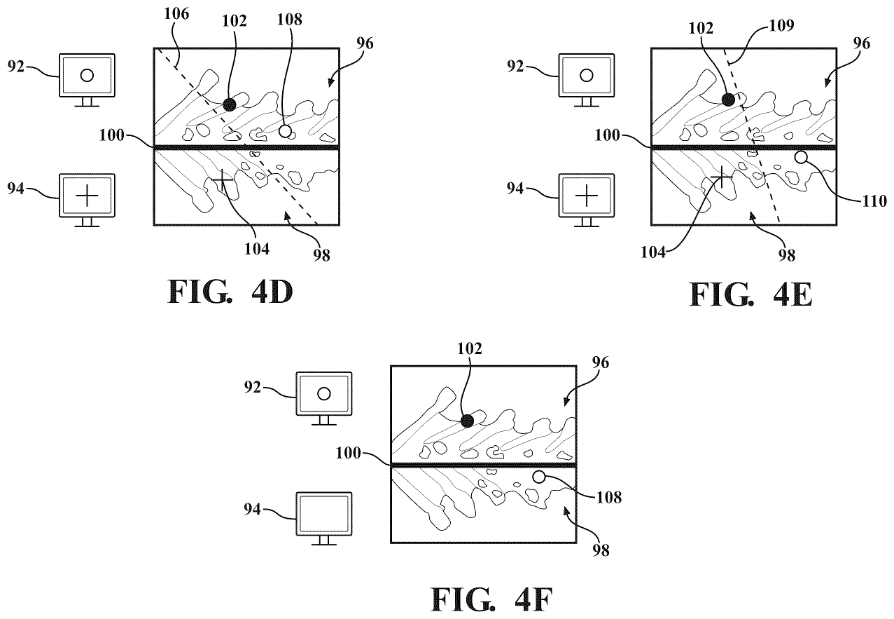

As previously described, the feedback parameters may define visual feedback, and the visual feedback may include the display of the navigation views 72, 74 from various perspectives/orientations. The navigation views 72, 74, as well as the various perspectives thereof, are described in reference to FIGS. 5-14B below.

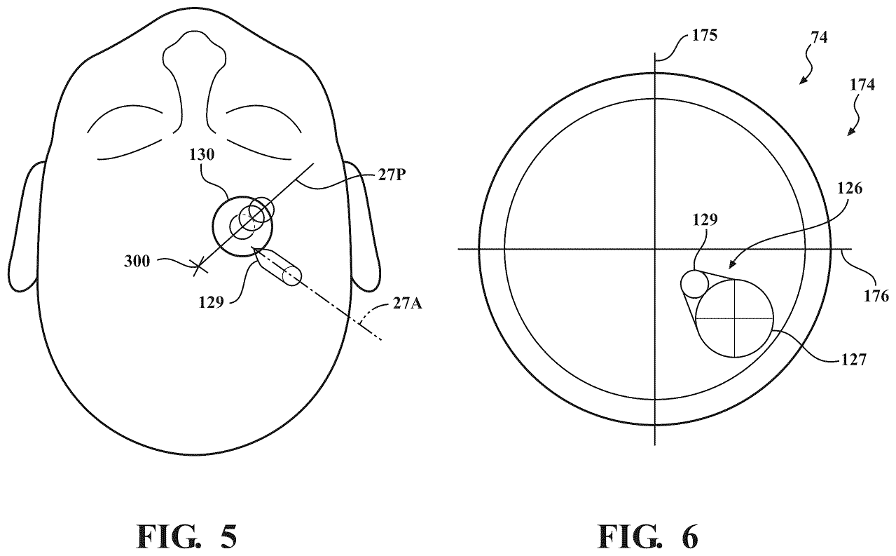

Referring to FIGS. 5 through 9, the navigation view 74 is shown in detail. The navigation view 74 does not necessarily include representations of the patient but instead includes a navigation indicator 174. To explain the operation of the navigation indicator 174, representations of the patient and the instrument are shown in FIGS. 5, 7, and 9, and corresponding operation of the navigation indicator 174 is shown in FIGS. 6, 8, and 10.

Starting with FIG. 5, a perspective view of a computer model representative of the patient (e.g., a three-dimensional model created based on two/three-dimensional image data) in which a planned trajectory 27P and a current trajectory 27A of the medical instrument is shown. A representation 129 of the current position of the distal tip 29 of the instrument 26 is also shown. The planned trajectory may include a planned entry point and/or a planned target point 300. The planned entry point may be visualized by an intersection between the planned trajectory 27P and a circular plane 130 tangential to an outer surface of the computer model, and the anatomical target 300 is shown at a terminating end of the planned trajectory 27P. As can be seen, the distal tip 29 of the medical instrument 26 is remote from the planned trajectory 27P and thus also remote from the planned target point 300. In addition, there is a large angle between the current trajectory 27A of the instrument 26 and the planned trajectory 27P.

FIG. 6 shows an example of the navigation view 74 with the medical instrument 24 being in the pose shown in FIG. 5. The navigation view is in this case determined by the computing system 4 based on the tracking data (i.e., the pose of the medical instrument 26). In the illustrated implementation, the navigation view 74 includes the navigation indicator 174, and the indicator 174 includes a pair of crosshairs 175, 176 and a representation 126 of the instrument 26. The crosshairs 175, 176 are representative of the planned trajectory 27P. The representation 126 of the instrument 26 may include the representation 129 of the distal tip 29 as well as a representation 127 of the shaft 27 of the instrument 26. The navigation indicator 174 is aligned with the planned trajectory 27P, and the crosshairs 175, 176 intersecting at a center of the navigation indicator 174 are representative of the planned trajectory 27P. The navigation indicator 174 may be like the bullseye indicator described in U.S. Patent Pub. No. 2021/0212767, entitled, “Technique of Controlling Display of a Navigation View Indicating an Instantaneously Changing Recommended Entry Point,” which is hereby incorporated by reference.

In order to show the pose of the instrument 26 (i.e., the current trajectory 27A) relative to the planned trajectory 27P (i.e., the pose of the actual trajectory 27A relative to the planned trajectory 27P), the representation 129 of the tip 29 of the instrument may be shown relative to the intersection of the crosshairs 175, 176, and the representations 127, 129 of the shaft and tip 27, 29 of the instrument 26 may be shown relative to one another. When the instrument 26 is following the planned trajectory 27P such that the current trajectory 27A is substantially in line with the planned trajectory 27P, the tip representation 129 overlaps the intersection of the crosshairs 175, 176, and the shaft representation 127 is centered on the tip representation 129. In the scenario shown by FIGS. 5 and 6, the navigation indicator 174 indicates that the instrument 26 should be (1) moved up and to the left relative to the surgeon and (2) tilted upwards and to the left relative to the surgeon. As described below, it is often important to reorient the navigation indicator 174 based on the position of the surgeon (e.g., based on the approach direction of the instrument 26)

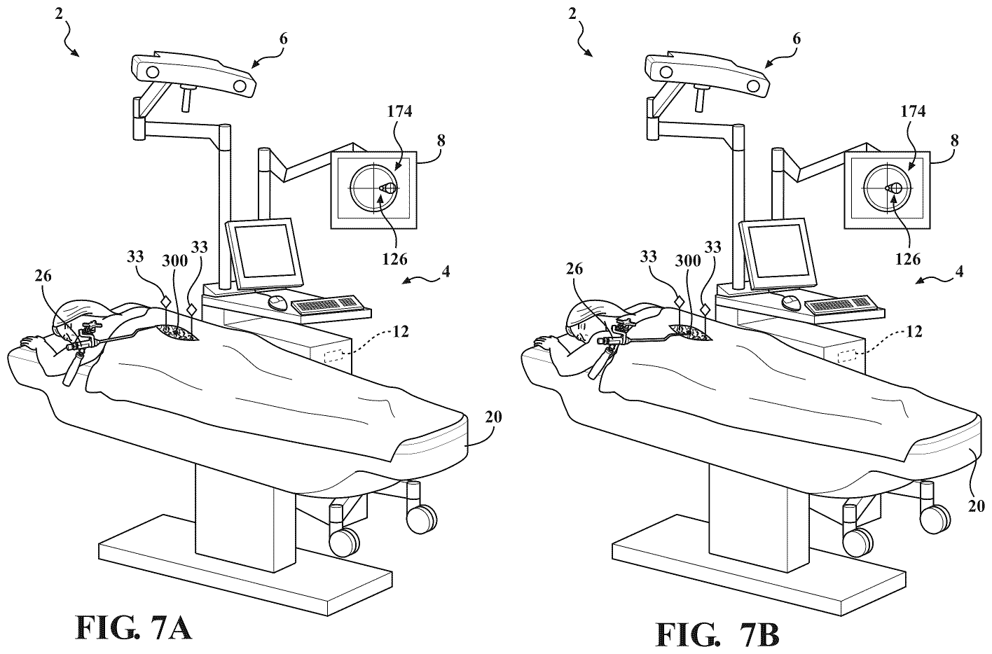

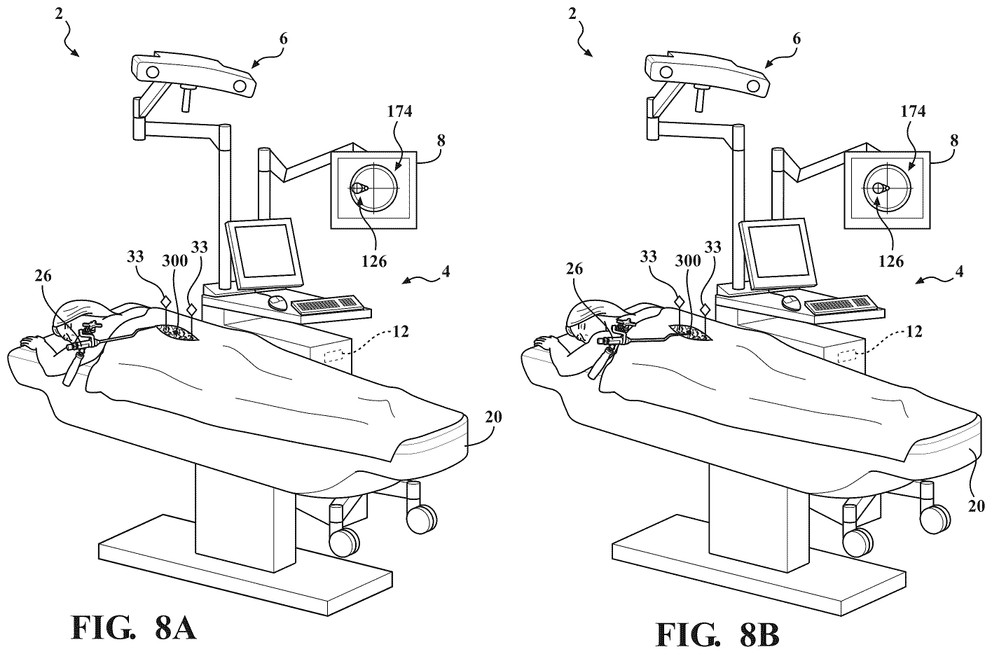

Referring to FIGS. 7A through 8B, the navigation indicator 74 is shown switching from a first perspective to a second perspective based on the position of the instrument 26. To illustrate the differences between the first perspective and the second perspective, the instrument 26 is shown moving to the right relative to the patient in FIGS. 7A and 7B and FIGS. 8A and 8B. In the first perspective, as shown in FIGS. 7A and 7B, the navigation indicator 174 indicates that the instrument 26 is to the right of an anatomical target 300 and that moving the instrument 26 to the left would align the instrument 26 with the anatomical target 300. Comparatively, in the second perspective shown in FIGS. 8A and 8B, the navigation indicator 174 indicates that the instrument 26 is to the left of the anatomical target 300 and that moving the instrument 26 to the right would align the instrument 26 with the anatomical target 300.

The preferred perspective of the navigation indicator 174 may depend on the location of the surgeon and/or the instrument 26. For example, if the surgeon is standing on the same side of the patient as the instrument 26 is positioned in FIGS. 7A through 8B, the second perspective may be preferred and shown on the display unit(s) 8. This is because the instrument 26 is shown as being to the left of the anatomical target 300 in second perspective of the navigation indicator 174, which matches the perspective of the patient as seen by the surgeon. Thus, the surgeon would need to simply observe the navigation indicator 174, see that the navigation indicator 174 indicates that the instrument 26 is to the left of the anatomical target 300, and understand that the instrument 26 must be moved to the right in order to align the current trajectory 27A of the instrument 26 with the planned trajectory 27P without needing to compensate for the differences between the surgeon's perspective of the patient and the perspective of the navigation indicator 174. In other words, if the navigation indicator 174 was shown in the first perspective like in FIGS. 7A and 7B, the surgeon would need to understand that moving the instrument 26 to their right would result in a leftward motion of the instrument 26 shown in the navigation indicator 174. As a result, the surgeon would be required to perform a mental transformation between their perspective of the patient and the perspective of the patient shown by the navigation indicator 174. Since requiring the surgeon to perform such mental transformations during an operation can take the attention of the surgeon away from other important tasks, it is advantageous to switch the perspective of the navigation indicator 174 to better align with the perspective of the surgeon, such as according to the methods described herein.

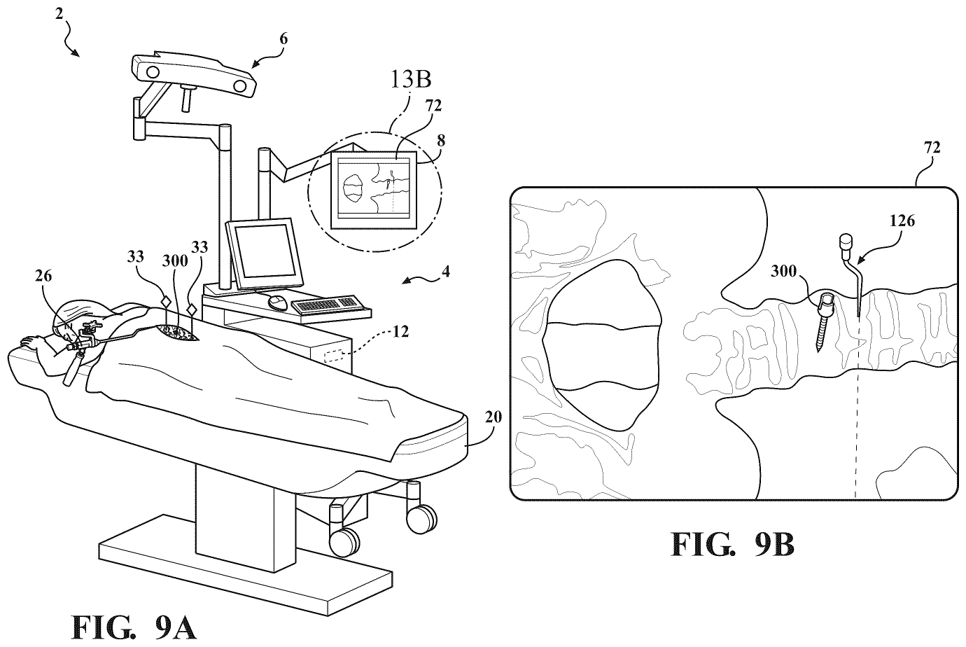

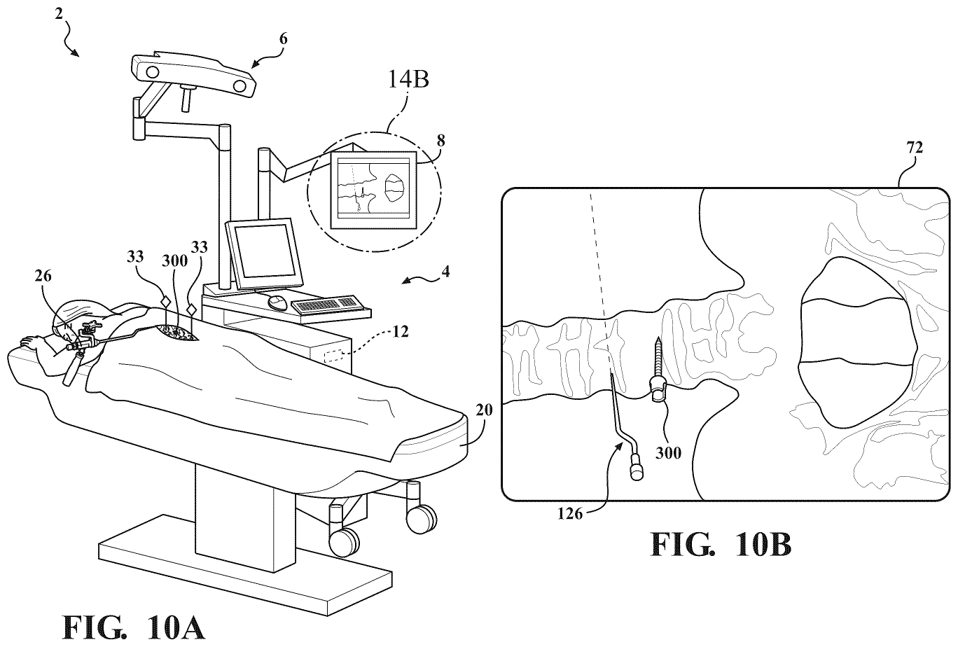

Referring to FIGS. 9A through 10B, the navigation view 72 is shown switching from a first perspective to a second perspective based on the position of the instrument 26, similar to FIGS. 7A through 8B for the navigation view 74. In the first navigation view perspective, the instrument 26 is depicted as being to the right of the anatomical target 300 (e.g., a pedicle screw). In the second navigation view perspective, however, the instrument 26 is shown as being to the left of the anatomical target 300. Depending on the perspective of the navigation view 72, the surgeon may be caused to move the instrument 26 in different directions or perform mental transformations to ensure that the instrument 26 is moved in the correct direction. For example, in the illustrated implementation, the navigation view 72 includes a representation of anatomy of the patient in the operating room, and the surgeon may be able to determine that the instrument should be moved towards a pelvis of the patient regardless of the perspective of the navigation view 72. Even though the surgeon may be able to transform the perspective of the navigation view 72 according to the surgeon's visual perspective of the patient, this can be mentally taxing for the surgeon. Therefore, adjusting the perspective of the navigation view 72 based on the position of the surgeon and/or instrument 26 is advantageous.

The perspective of the navigation indicator 174 and/or the navigation view 72 may be changed based on the position/pose of the instrument 26. Updating the perspective based on the pose/position of the instrument 26 may be performed based on the spatial regions and/or virtual planes 100 as described above. For example, the perspective may be based on at least one of a maximum distance between the instrument and the spatial region, a maximum distance between the instrument and an anatomical element enclosed by and/or defining the spatial region, a maximum deviation of a main instrument axis from the spatial region, and a maximum deviation of a main instrument axis from an anatomical element enclosed by and/or defining the spatial region.

Additionally or alternatively, the perspective of the navigation indicator 174 and/or the navigation view 72 may be updated based on an approach direction of the instrument 26. Methods of updating the navigation indicator 174 and the navigation view 72 are described below. The methods are described as being carried out by the computing system 4, which may include the at least one processor 12 and the at least one memory 14, but the methods may be carried out by other computing devices, such as cloud computing devices. The methods are also described in reference to the instrument 26, but may also be carried out based on any combination of the instruments 24, 26, 28 and/or multiple displays 8. For example, a first display may be associated with the instrument 24, a second display may be associated with the instrument 26, and the navigation indicator 174 and/or navigation view 72 may be updated on the displays based on the approach direction of the associated instrument 24, 26, 28.

Starting with the navigation indicator 174, in one implementation, the method may begin with obtaining a computer model representative of at least a portion of the patient present in the operating room, planning data including a planned trajectory (e.g., a planned entry point and a planned anatomical target), spatial information, and tracking information. The spatial information may be like that described above and may be indicative of a first spatial region associated with a first side of the patient and a second spatial region associated with a second side of the patient. The tracking information may also be like that described above and may be indicative of the pose of the instrument 26. The tracking information may further include past poses/positions of the instrument 26 to enable the computing system 4 to determine an approach direction of the instrument 26. The spatial information and/or the tracking information may be defined/known relative to a coordinate system associated with the computer model representative of at least a portion of the patient.

Once the computer model, planning data, spatial information, and tracking information have been obtained, the computing system 4 may determine a current trajectory of the instrument 26 based on the tracking information. The current trajectory may include a current entry point that represents an approximation of where the instrument 26 will enter the anatomy and is based on at least one of the distal tip 29 of the instrument 26 and the shaft 27 of the instrument 26. Then, the computing system 4 may determine the approach direction of the instrument 26 based on the tracking information. In some implementations, the approach direction is only determined once the instrument 26 is within a threshold distance of the anatomical target 300. In either case, the perspective of the navigation indicator 174 may be set by the computing system 4 based on the approach direction of the instrument 26 once the approach direction is determined. For example, looking at FIGS. 7A through 8B, the instrument 26 is shown as approaching from the left side of the patient while the display 8 is arranged on the right side of the patient. As such, the navigation indicator 174 is adjusted in FIGS. 8A and 8B, relative to FIGS. 7A and 7B, to align with the surgeon's perspective of the patient. Instead, if the instrument 26 (and, therefore, surgeon) were on the right side of the patient and the display 8 was on the left side of the patient, the perspective of the navigation indicator 174 may be set like that shown in FIGS. 7A and 7B.

As for the navigation view 72, in one implementation, the method may start similarly to the method of adjusting the perspective of the navigation indicator 174. More specifically, the method may start with obtaining the spatial information, the tracking information, and image data representative of at least a portion of a patient in the operating room. Again, the spatial information and tracking information may be like that described above. Once the spatial information, tracking information, and image data have been obtained, the computing system 4 may determine the approach direction of the instrument 26 based on the spatial information and the tracking information. In some implementations, the approach direction is only determined once the instrument 26 is within a threshold distance of the anatomical target 300. Subsequently, the computing system 4 may select a navigation view perspective based on the approach direction of the instrument 26. Finally, the perspective of the navigation view 72 may be set/displayed according to the selected navigation view perspective.

In one implementation, the navigation view perspective may be displayed like those shown in FIGS. 9A through 10B. For example, if the instrument 26 is approaching from the left side of the patient like shown in FIGS. 9A and 10A, the perspective of the navigation view 72 may be set/displayed like that shown in FIG. 10B. As can be appreciated from the figures, the pelvis is shown to the left of the anatomical target 300 in the navigation view perspective shown in FIG. 9B, however, the pelvis of the patient would be to the right of the anatomical target 300 relative to a surgeon standing on the left side of the patient (e.g., holding the instrument 26 at the pose shown in FIGS. 9A and 10A. Thus, the perspective of the navigation view 72 may be switched to be like that shown in FIG. 10B. This way, the orientation of the image data of the patient is aligned to the surgeon's perspective of the patient so that a rightward motion of the instrument 26 (i.e., moving away from the head of the patient and towards the feet of the patient) is shown as a rightward motion of the instrument representation 126.

In addition to reorienting the representation of the image data when changing the perspective of the navigation view 72, the representation of the instrument 26 may also be changed to align with the surgeon's perspective of the instrument 26. For example, as shown in FIGS. 9B and 10B, the representation 126 of the instrument 26 is overlaid in different orientations based on the selected/displayed perspective of the navigation view 72, while the overall shape of the representation 126 is unchanged. In the navigation view perspective of FIG. 9B, the representation 126 is overlaid in a first orientation relative to the display 8. And in the navigation view perspective of FIG. 10B, the representation 126 is overlaid in a second orientation relative to the display 8.

The representation 126 of the instrument 26 is shown as a two-dimensional graphic in the figures, but may instead be a three-dimensional rendering that includes a profile and a contour. The profile may define the overall shape/outline of the three-dimensional rendering, while the contour may give the representation 126 an appearance of facing into the display 8 or out of the display 9. The computing system 4 may be configured to adjust the profile and/or shape of the rendering so that the representation 126 shown on the display 8 is similar in appearance to the instrument 26 from the surgeon's perspective. In some implementations, the rendering 126 has the same profile regardless of the selected navigation view perspective, but the contour is changed based on the selected navigation view perspective. In other implementations, both the profile and the contour are changed based on the selected navigation view perspective.

During the methods described above, the approach direction of the instrument 26 may be determined in various ways. In one implementation, the tracking information may include a first position of the instrument 26 corresponding to the position of the instrument 26 at a first time and a second position of the instrument 26 corresponding to the position of the instrument 26 at a second time. In this implementation, the approach direction of the instrument 26 may be determined by comparing the second position of the instrument 26 to the first position of the instrument 26. This may include determining a direction of movement relative to a coordinate system, such as the real world coordinate system or coordinate system associated with the tracking system 6. In another implementation, the computing system 4 may determine that the instrument 26 is moving towards the anatomical target 300 while also being present within one of the spatial zones. The computing system 4 may then determine that the instrument 26 is approaching from the side of the patient that is associated with the spatial zone through which the instrument 26 is moving. For example, the spatial zones may be separated by the virtual plane 86 shown in FIG. 3E, and the computing system 4 may determine if the instrument 26 is on the left or right side of the patient based on the spatial relationship between the instrument 26 and the virtual plane 86. In yet another implementation, the computing system 4 may determine which spatial zones the instrument 26 recently traveled through before reaching its current pose to determine the approach direction. For example, a patient zone surrounding the patient may be adjacent to a left zone, arranged to the left of the patient, and a right zone, arranged to the right of the patient. In this example, the computing system 4 may determine in which of the left/right zones the instrument 26 was present prior to entering the patient zone.

Several embodiments have been discussed in the foregoing description. However, the implementations discussed herein are not intended to be exhaustive or limit the filter assembly to any particular form factor. The terminology which has been used is intended to be in the nature of words of description rather than of limitation. Many modifications and variations are possible in light of the above teachings and the system may be practiced otherwise than as specifically described.

1. A computer-implemented method for supporting users in an operating room, the method being performed by at least one processor and comprising:
obtaining spatial information indicative of a plurality of spatial regions in the operating room;
obtaining tracking information indicative of a tracked pose of a medical instrument in the operating room;
obtaining one or more predefined spatial constraints for one or more of the plurality of spatial regions;
determining, based on the spatial information and the tracking information, whether the one or more predefined spatial constraints are met;
associating the medical instrument with one of the plurality of spatial regions based on whether the one or more predefined spatial constraints are met; and
triggering visual feedback according to one or more feedback parameters of the spatial region associated with the medical instrument, wherein the one or more feedback parameters define a perspective of a navigation view being displayed to the user.

2. The computer-implemented method of claim 1, wherein the one or more predefined spatial constraints comprise at least one of:
a maximum distance between the instrument and the spatial region,
a maximum distance between the instrument and an anatomical element enclosed by and/or defining the spatial region,
a maximum deviation of a main instrument axis from the spatial region, and
a maximum deviation of a main instrument axis from an anatomical element enclosed by and/or defining the spatial region.

3. The computer-implemented method of claim 1, wherein at least one of the spatial information and the tracking information is obtained for multiple points in time and the visual feedback is iteratively triggered for two or more of the multiple points in time.

4. The computer-implemented method of claim 1, wherein the plurality of spatial regions are separated from one another by one or more virtual planes defined relative to a patient.

5. The computer-implemented method of claim 1, wherein the spatial information is determined or defined based on patient image data comprising one or more medical images of at least a portion of a patient's body.

6. A computer-implemented method for supporting users in an operating room, the method being performed by at least one processor and comprising:
obtaining a computer model representative of at least a portion of a patient in the operating room;
obtaining planning data including a planned trajectory defined relative to a coordinate system associated with the computer model;
obtaining spatial information indicative of a first spatial region associated with a first side of the patient and a second spatial region associated with a second side of the patient;
obtaining tracking information associated with a medical instrument;
determining, based on the tracking information, a current trajectory of the medical instrument relative to the coordinate system associated with the computer model;
determining, based on the spatial information and tracking information, whether the medical instrument is approaching the patient from the first side or the second side; and
controlling display of a navigation indicator visualizing a spatial relationship between the planned trajectory and the current trajectory based on whether the medical instrument is approaching the patient from the first side or the second side.

7. The computer-implemented method of claim 6, wherein the planned trajectory includes a planned entry point and a planned target.

8. The computer-implemented method of claim 6, wherein the spatial information is defined relative to the coordinate system associated with the computer model.

9. The computer-implemented method of claim 6, further comprising:
causing the navigation indicator to visualize a first direction of movement of the medical instrument as a first direction of movement of the current trajectory of the medical instrument relative to the planned trajectory in response to the medical instrument approaching the patient from the first side; and
causing the navigation indicator to visualize the first direction of movement of the medical instrument as a second direction of movement of the current trajectory of the medical instrument relative to the planned trajectory in response to the medical instrument approaching the patient from the second side.

10. The computer-implemented method of claim 6, wherein the current trajectory includes at least one of a current position of a distal tip of the medical instrument and a current position of a shaft of the medical instrument.

11. The computer-implemented method of claim 10, wherein:
the navigation indicator includes crosshairs representative of the planned trajectory and a representation of the current position of the distal tip of the medical instrument; and
the method further comprises:
causing the representation of the current position of the distal tip of the medical instrument to move in a first direction relative to the crosshairs in response to the medical instrument being moved towards the planned trajectory and the medical instrument approaching the patient from the first side; and
causing the representation of the current position of the distal tip of the medical instrument to move in a second direction relative to the crosshairs in response to the distal tip of the medical instrument being moved towards the planned trajectory and the medical instrument approaching the patient from the second side.

12. The computer-implemented method of claim 11, wherein:
the navigation indicator further includes a representation of the current position of the shaft of the medical instrument; and
the method further comprises:
causing the representation of the current position of the shaft of the medical instrument to move in a first direction relative to the representation of the current position of the distal tip of the medical instrument in response to the medical instrument being tilted towards the planned trajectory and the medical instrument approaching the patient from the first side; and
causing the representation of the current position of the shaft of the medical instrument to move in a second direction relative to the representation of the current position of the distal tip of the medical instrument in response to the medical instrument being tilted towards the planned trajectory and the medical instrument approaching the patient from the second side.

13. The computer-implemented method of claim 6, wherein the display of the navigation indicator is controlled in response to the medical instrument being within a threshold distance of an anatomical target.

14. The computer-implemented method of claim 6, wherein:
the tracking information includes a first position of the medical instrument corresponding to a first time and a second position of the medical instrument corresponding to a second time; and
the computer-implemented method further comprises:
calculating an approach direction of the medical instrument based on the first and second positions of the medical instrument; and
determining whether the medical instrument is approaching the patient from the first side or the second side based on the calculated approach direction.

15. A computer-implemented method for supporting users in an operating room, the method being performed by at least one processor and comprising:
obtaining image data representative of at least a portion of a patient in the operating room;
obtaining spatial information indicative of a first spatial region associated with a first side of the patient in the operating room and a second spatial region associated with a second side of the patient in the operating room;
obtaining tracking information associated with a medical instrument in the operating room;
determining, based on the spatial information and tracking information, whether the medical instrument is approaching the patient from the first side or the second side;
selecting (i) a first navigation view perspective in response to the medical instrument approaching the patient from the first side, or (ii) a second navigation view perspective in response to the medical instrument approaching the patient from the second side; and
displaying a representation of the image data based on the selected navigation view perspective on a display.

16. The computer-implemented method of claim 15, wherein the first navigation view perspective is mirrored relative to the second navigation view perspective.

17. The computer-implemented method of claim 15, further comprising:
overlaying a representation of the medical instrument over the representation of the image data based on the image data and the tracking information; and
determining that the medical instrument is moving towards an anatomical target;
wherein the overlaid representation of the medical instrument is displayed as moving towards the anatomical target and (i) in a first direction relative to the user when the first navigation view perspective is selected, or (ii) in a second direction relative to the user when the second navigation view perspective is selected.

18. The computer-implemented method of claim 17, wherein:
the representation of the medical instrument includes a rendering of the medical instrument; and
the rendering of the medical instrument is overlaid over the representation of the image data based on the selected navigation view perspective.

19. The computer-implemented method of claim 18, wherein:
the rendering of the medical instrument includes a shape and an orientation;
the rendering of the medical instrument has the same shape when either of the first and second navigation view perspectives are selected; and
the orientation of the rendering of the medical instrument relative to the display is changed based on the selected navigation view perspective.

20. The computer-implemented method of claim 19, wherein the orientation of the rendering of the medical instrument relative to the representation of the image data is not changed based on the selected navigation view perspective.

21. The computer-implemented method of claim 19, wherein:
the rendering of the medical instrument is a three-dimensional rendering including a profile and a contour;
the rendering of the medical instrument has the same profile when either of the first and second navigation view perspectives are selected; and
the contour of the rendering of the medical instrument relative to the display is changed based on selected navigation view perspective.

22. The computer-implemented method of claim 15, wherein the navigation view perspective is selected in response to the medical instrument being within a threshold distance of an anatomical target.

23. The computer-implemented method of claim 16, wherein:
the tracking information includes a first position of the medical instrument corresponding to a first time and a second position of the medical instrument corresponding to a second time; and
the computer-implemented method further comprises:
calculating an approach direction of the medical instrument based on the first and second positions of the medical instrument; and
determining whether the medical instrument is approaching the patient from the first side or the second side based on the calculated approach direction.
